(19) United States
(12) Patent Application Publication
(10) Pub. No.: US 2023/0398163 A1
(43) Pub. Date: Dec. 14, 2023

(54) FRUIT EXTRACTS FOR USE IN THE TREATMENT OF NEURODEGENERATIVE DISEASES

(71) Applicant: NEUROSCIENCE PHARMA SPA, Temuco (CL)

(72) Inventors: Sebastian MONCKEBERG VERGARA, Temuco (CL); Jorge Patricio FUENTEALBA ARCOS, Temuco (CL)

(73) Assignee: NEUROSCIENCE PHARMA SPA, Temuco (CL)

(21) Appl. No.: 18/022,877

(22) PCT Filed: Aug. 24, 2021
(86) PCT No.: PCT/IB2021/057735

§ 371 Date: Feb. 23, 2023

Related U.S. Application Data
(60) Provisional application No. 63/069,534, filed on Aug. 24, 2020.

(51) Int. Cl. A61K 36/185; A61K 9/00; A23G 3/48

(52) U.S. Cl. CPC A61K 36/185; A61K 2236/51; A23G 3/48; A61K 9/0056

(57) ABSTRACT

Fruit and berry extracts, including maqui berry extracts, are described herein. Also described are methods of making such fruit and berry extracts, including maqui berry extracts. In certain embodiments, the extract may be made by separating fruit or berries into a polyphenol-containing liquid fraction and a fiber-containing solid fraction; concentrating the liquid fraction to form a concentrated liquid fraction; combining the concentrated liquid fraction with a solid carrier to form a mixture; and drying the mixture to form the extract. The extracts may be used to treat, or prevent or delay the onset of, certain neurodegenerative diseases, such as Alzheimer's disease or Parkinson's disease.

### DESCRIPTIVE MEMORY

This application claims priority benefit of United States Provisional Patent Application No. 63/069,534 filed on Aug. 24, 2020, the contents of which are incorporated herein by reference in their entirety.

### FIELD OF THE INVENTION

Fruit extracts, including maqui berry extracts, and methods for manufacturing fruit extracts are described herein. Also described are methods for treating a neurodegenerative disease, such as Alzheimer's disease, using the fruit extracts.

### BACKGROUND

Alzheimer's disease (AD) is a complex and progressive clinical condition where the main consequences are related to cognitive and memory dysfunctions due to irreversible neurodegeneration, synaptic dysfunction, and neuronal death. It is currently believed that AD is a multifaceted disease characterized by an abnormal increase in amyloid-β peptide (Aβ) levels. These increased levels have been related to different hypotheses trying to explain the physiopathology of the disease, for example: AβPP mutation, mutations in the presenilin gene 1 and gene 2, and others. All these factors can increase Aβ levels and appear to be important in the onset of toxic effects and could be considered a target for the development of new drugs or treatment strategies to slow or stop the progression of the disease.

AD continues to be a biomedical challenge due to the absence of effective therapies that could deter the progression of the disease, and also due to the lack of biomarkers that would allow for an early detection. Therefore, any clinical, nutraceutical, or pharmacological strategy that could help to slow down disease advancement or be considered as a parallel treatment would be a significant alternative for these patients.

Recent studies have demonstrated the foods high polyphenol content are useful in slowing the progression of certain neurological diseases. See, for example, Weinreb et al., Neurological mechanisms of green tea polyphenols in Alzheimer's and Parkinson's disease, J. Nutritional Biochemistry, vol. 15, pp. 506-516 (2004); Rossi et al., Benefits from Dietary Polyphenols for Brain Aging and Alzheimer's Disease, Neurochem Res., vol. 33 pp. 2390-2400 (2008). Maqui berries are the fruiting bodies of the maqui tree (Aristotelia chilensis), native to Chile. The berries have been found to have a high polyphenol content, particularly anthocyanin content. However, polyphenols are relatively unstable compounds that can degrade during an extraction process. For example, a standard freeze-drying process of maqui berries can result in polyphenol decay of over 60%.

### BRIEF SUMMARY OF THE INVENTION

For effective treatment of neurodegenerative diseases, including Alzheimer's disease, maqui berry extracts should contain a high polyphenol content. Additionally, methods of making the maqui berry extracts should be cost effective. Described herein are maqui berry extracts with high polyphenol, including high anthocyanin content. The maqui berry extracts are manufactured using cost-effective methods, which can allow for a reasonably priced product useful for treating neurodegenerative disease.

In some embodiments, the method of making the fruit or berry extract includes separating a fruit or berries into a polyphenol-containing liquid fraction and a fiber-containing solid fraction; concentrating the liquid fraction to form a concentrated liquid fraction; combining the concentrated liquid fraction with a solid carrier to form a mixture; and drying the mixture to form the extract. In some embodiments, the fruit or berries are maqui berries.

In some embodiments, the fiber-containing solid fraction includes polyphenols from the fruit or berries. The amount of polyphenols in the fiber-containing solid fraction may be about 50% or more of the polyphenols from the fruit of berries. In other variants of the method, the polyphenol-containing liquid fraction includes about 50% or more of the polyphenols from the fruit or berries.

In some embodiments, the solid carrier comprises a portion or all of the fiber-containing solid fraction. In some embodiments, the method includes drying the solid fraction prior to mixing the concentrated liquid fraction with the solid carrier. The solid fraction may be dried, for example under a vacuum pressure. The solid fraction may be dried, for example, at a temperature between about −30° C. and about 30° C., or about −20° C. and about 20° C.

In some embodiments, concentrating the liquid fraction comprises applying a vacuum and a secondary power to the liquid fraction. In some embodiments, the secondary power applied to the liquid fraction comprises microwave power. In some embodiments, the secondary power applied to the liquid fraction is heat by conduction.

The illiquid fraction may be maintained at a temperature above freezing, for example about 10° C. to about 30° C., while being concentrated.

The liquid fraction may be concentrated, for example, using a continuous evaporator that simultaneously receives the liquid fraction and outflows the concentrated liquid fraction. The liquid fraction in the continuous evaporator may have, for example, a soluble solids content of about 50% to about 80%.

In some embodiments, the method of making the fruit or berry extract includes removing seeds or seed pieces prior to separating the fruit or berry prior to separating the fruit or berries into the liquid fraction and the solid fraction.

In some embodiments, the method of making the fruit or berry extract includes pulping fruit or berries to form fruit or berry pulp, wherein the liquid fraction and the solid fraction are formed from the fruit or berry pulp. In some embodiments, the method includes deactivating endogenous enzymes in the fruit or berry pulp. In some embodiments, deactivating the endogenous enzymes includes heating the fruit or berry pulp to a temperature between about 60° C. to about 100° C., and cooling the fruit or berry pulp. In some embodiments, the method includes applying an ultrasonic, microwave energy, or an electromagnetic energy to the pulp. In some embodiments, the method includes mixing the fruit or berry pulp with one or more enzymes that at least partially degrade fiber in the fruit or berry pulp. In some embodiments, the one or more enzymes comprises cellulase, pectinase, or hemicellulase.

In some embodiments, the method of making the fruit or berry extract includes pelleting the mixture prior to drying the mixture.

In some embodiments, the method of making the fruit or berry extract includes forming a layer comprising the mixture, wherein the layer has a thickness of about 1 mm to about 5 mm.

In some embodiments, the method of making the fruit or berry extract includes freezing the mixture prior to drying the mixture

In some embodiments of the method of making the fruit or berry extract, the method includes drying the mixture comprises applying a vacuum and a secondary power to the mixture.

In some embodiments, the secondary power is applied using infrared, temperature differential radiation, direct conduction, or microwave. In some embodiments, the secondary power is applied using microwave. In some embodiments, the power is applied at about 0.05 kW to about 0.25 kW per kg of the mixture.

In some embodiments of the method of making the fruit or berry extract, the method includes pelleting the mixture to form a pelleted mixture: freezing the pelleted mixture to form a frozen, pelleted mixture; and drying the frozen, pelleted mixture by applying a vacuum and microwave power to the frozen, pelleted mixture, to form the extract. In some embodiments, the microwave power is applied at about 0.05 kW to about 0.25 kW per kg of the mixture.

In some embodiments of the method of making the fruit or berry extract, the method includes forming a layer comprising the mixture, wherein the layer has a thickness of about 1 mm to about 5 mm; and drying the layer comprising the mixture by applying a vacuum and a secondary power to the layer, to form the extract. In some embodiments, the secondary power is applied using infrared, temperature differential radiation, or direct conduction. In some embodiments, the secondary power is applied at about 0.05 kW to about 0.25 kW per kg of the mixture.

In some embodiments of the method of making the fruit or berry extract, the temperature of the mixture increases to a maximum temperature of 50° C. or less during the drying.

In some embodiments of the method of making the fruit or berry extract, the temperature of the mixture increases as the water activity of mixture decreases during the drying.

In some embodiments of the method of making the fruit or berry extract, the temperature of the mixture does not exceed 30° C. for more than about 30 minutes during the drying.

In some embodiments of the method of making the fruit or berry extract, the liquid fraction is concentrated at a temperature between about 20° C. and about 35° C. when the water activity of the liquid fraction is about 0.75.

In some embodiments of the method of making the fruit or berry extract, the liquid fraction is concentrated at a temperature between about 30° C. and about 45° C. when the water activity of the liquid fraction is between about 0.6 and about 0.75.

In some embodiments of the method of making the fruit or berry extract, the fruit or berries are separated into the liquid fraction and the solid fraction using centrifugation or counter-current extraction. In another example, the fruit or berries are separated into the liquid fraction and the solid fraction by pressing fruit or berry pulp.

In some embodiments of the method of making the fruit or berry extract, the mixture is dried until it has a moisture content of about 5% or less by weight.

In some embodiments of the method of making the fruit or berry extract, the method includes processing the extract into a powder.

Also described herein is a method of making a liquid fruit or berry (such as maqui berry) extract, which includes: separating a fruit or berries into a polyphenol-containing liquid fraction and a fiber-containing solid fraction; and concentrating the liquid fraction, comprising applying a vacuum and a secondary power to the liquid fraction, to form a concentrated liquid fruit or berry extract. In some embodiments, the secondary power applied to the liquid fraction is a microwave power. In some embodiments, the secondary power applied to the liquid fraction is heat by conduction. In some embodiments, the liquid fraction is maintained at a temperature above freezing, such as about 10° C. to about 30° C. while being concentrated. In some embodiments, the liquid fraction is concentrated using a continuous evaporator that simultaneously receives the liquid fraction and outflows the concentrated liquid fraction. Liquid fraction in the continuous evaporator may have, for example, a soluble solids content of about 50% to about 80%. In some embodiments, concentrating the liquid fraction comprises a first concentrating step, comprising applying the vacuum to the liquid fraction without applying the secondary power to the liquid fraction; and a second concentrating step, comprising applying the vacuum and the secondary power to the liquid fraction. For example, in some embodiments, concentrating the liquid fraction comprises a first concentrating step, comprising applying the vacuum to the liquid fraction without applying microwave power to the liquid fraction; and a second concentrating step, comprising applying the vacuum and microwave power to the liquid fraction.

Also described herein is a fruit or berry extract made by any one of the methods described above.

Further described is a method of making a pharmaceutical or nutraceutical composition, which includes formulating the extract with one or more pharmaceutically acceptable or neutracetucially acceptable excipients. Also described is said pharmaceutical composition or a nutraceutical composition.

In some embodiments, the maqui berry extract includes at least 2% total polyphenol content by weight, at least 10% maqui berry fiber by weight, and a moisture content of less than 5% by weight. In some embodiments, the maqui berry extract includes at least 10% total polyphenol content by weight. In some embodiments, the maqui berry extract includes about 14% to about 40% total polyphenol content by weight. In some embodiments, the maqui berry extract includes at least 25% maqui berry fiber by weight. In some embodiments, the maqui berry fiber includes insoluble maqui berry fiber. In some embodiments, the maqui berry fiber includes soluble maqui berry fiber.

In some embodiments, the maqui berry extract is a powder.

In some embodiments, the maqui berry extract is substantially free of maqui seeds or maqui seed pieces.

Also described herein is a pharmaceutical or nutracentical dosage form, comprising the fruit or berry (e.g., maqui berry) extract, wherein the dosage form is formulated as an oral dosage form. In some embodiments, the oral dosage form is a chewable dosage form or a capsule. In some embodiments, the oral dosage form is a chewable gummy.

Also described is a method of treating a neurodegenerative disease in a subject, comprising administering to the subject a therapeutically effective amount of the extract described above, the composition described above, or the pharmaceutical dosage form described above.

Furthered described is a method of preventing or delaying the onset of a neurodegenerative disease in a subject, comprising administering to the subject a therapeutically effective amount of the extract described above, the composition described above, or the pharmaceutical dosage form described above. In some embodiments, the method further includes administering to the subject a therapeutically effective amount of an additional therapeutic agent for treating, preventing, or delaying the onset of the neurodegenerative disease. In some embodiments, the neurodegenerative disease is Alzheimer's disease or Parkinson's disease.

Also described is a method of increasing memory of a subject, comprising administering to the subject an effective amount of the extract described above, the composition described above, or the pharmaceutical dosage form described above.

### BRIEF DESCRIPTION OF THE DRAWINGS

FIG. 1A shows an exemplary method of making a maqui berry extract, according to some embodiments.

FIG. 1B shows another exemplary method of making a maqui berry extract, according to some embodiments.

FIG. 1C shows another exemplary method of making a maqui berry extract, according to some embodiments.

FIG. 2 shows an exemplary system for deactivating endogenous enzymes in the berry or fruit pulp, according to some embodiments.

FIG. 3 shows an exemplary continuous evaporator for concentrating the liquid fraction in

accordance with some embodiments.

FIG. 4 shows detect amyloid beta (Aβ) aggregation in the presence of 0.81 mg/l maqui berry extract or 0.081 mg/l maqui berry extract, or in the absence of maqui berry extract. Increasing amounts of maqui berry extract lowers the amount of Aβ aggregate formation.

FIG. 5 shows cell viability assay (MTT assay) results in presence of up to 81 mg/l maqui berry extracts, which demonstrates maqui berry extract is not cytotoxic.

FIG. 6 shows cell viability assay (MTT assay) results in presence of Aβ with no or up to 81 mg/l maqui berry extracts. The data demonstrate that the Aβ aggregates are cytotoxic, but that this cytotoxicity can be mitigated using maqui berry extract.

FIG. 7 shows the neuron synaptic frequency measure as Ca²⁺ systolic transient signal recordings in a neurocampal cell array. “Control” represents the synaptic frequency in a standard hippocampal neuron array. “AB” shows the effects of the Aβ application (0.5 μM for 24 hours) on the synaptic rate, reducing the intensity and rate, also producing synaptic silence. “MBX” (maqui berry extract) shows the same results control with the only application of the maqui berry extract (0.081 mg/l), showing a synaptic frequency increase. MBX+AB shows the results of the maqui berry extract and Aβ application to the control base array. The presence of the maqui berry extract significantly reverses the toxic effect of Aβ over the neurological synaptic process.

FIG. 8 shows the quantification of the Ca²⁺ systolic evens in the hippocampal neuron array for 200 milliseconds period as shown in FIG. 6, which indicates that the maqui berry extract not only reverts the toxic effect in the synaptic process due to Aβ, but further promotes it compared to the control.

FIG. 9 shows immunohistochemistry images of hippocampal neuronal arrays treated with Aβ, treated with Aβ and maqui berry extract, or untreated (control), before being stained for SV2 and MAP2. SV2 indicates the presence of neuronal synapses, which were diminished in cells treated with Aβ, but recovered when cells were treated with both Aβ and maqui berry extract.

FIG. 10 shows the quantification number of SV2 positive connection scores for each primary process analyzed. The graph also reinforces the appreciation of the positive effect of the maqui berry extract over Aβ aggregates.

FIG. 11A shows a significant improvement in Barnes Maze, Short Term Memory Test, for mice treated with maqui berry extract.

FIG. 11B shows a significant improvement in Barnes Maze, Long Term Memory Test, for mice treated with maqui berry extract.

### DETAILED DESCRIPTION OF THE INVENTION

Maqui berry extracts with a high polyphenol content are described herein. Further described are method of making such maqui berry extracts, and other fruit or berry extracts with high polyphenol content, and methods of using such maqui berry extracts for treating neurodegenerative disorders (such as Alzheimer's disease). The maqui berry extract may be used to treat neurodegenerative diseases in combination with one or more additional therapeutic agents

Directly drying maqui berry pulp can result in degradation of the polyphenols if temperatures become too high for an extended period of time during the drying process. Polyphenols are more heat sensitive under higher water activity (a_{w}) levels (e.g., about 0.7 to about 0.95). Although the polyphenols can be readily extracted from the pulp in a liquid fraction, the high sugar content of the liquid fraction prevents drying without substantially increasing the temperature. As the liquid fraction is concentrated, the sugars cause the composition to enter into a glassy phase that retains water. Once the composition enters a glassy phase, it is very difficult, if not impossible, to further dry the composition without applying significant amounts of energy, resulting in polyphenol degradation. One solution is to remove sugars from the liquid fraction, but this process is generally expensive and time-consuming, which can result in further degradation of the polyphenols.

Methods described herein for making the fruit or berry (e.g., maqui berry) extract include separating a polyphenol-containing liquid fraction from fruit or berry pulp (and, in some embodiments, seeds of the fruit or berry), concentrating the liquid fraction, combining the concentrated liquid fraction with a solid, fibrous carrier (e.g., natural fibers from the fruit or berry), and drying the resulting semi-solid mixture to form a powder. As the semi-solid mixture, the temperature can be raised as the water activity decreases. For example, in some embodiments. about 70% to about 80% of the moisture may be removed at lower temperatures (e.g., below about 30° C.) until the water activity decreases to less than about 0.7. This process may take, for example, less than 2 hours, such as about 5 minutes to about 1 hour, or about 10 minutes to about 30 minutes. Once the water activity is low, the temperature can be increased (e.g., to between about 30° C. and about 50° C.) to further dry the composition. This portion of the process may occur in about 3 hours or less, such as about 2 hours or less, about 1 hour or less, or about 30 minutes or less. Using this process, a dried and powdered extract with high polyphenol content can be obtained.

As further described herein, the polyphenol-containing liquid fraction can be concentrated, but not completely dried, and then mixed with a solid and preferably fibrous carrier (for example, dried fibers extracted from the maqui berry pulp). The resulting mixture can be more easily dried at lower temperatures, which minimizes degradation of the polyphenols. This process is particularly advantageous because it avoids the need to remove the natural sugars and results in an extract with a particularly high polyphenol content. Further, the amount of fibrous carrier mixed with the concentrated liquid fraction can be controlled to minimize non-polyphenol “bulk” in the final extract.

The extracts described herein, which may be a liquid extract or a solid (e.g., powdered) extract, can be used for treatment of certain neurodegenerative diseases, such as Alzheimer's disease or Parkinson's disease. In some embodiments, the extract is formulated into a nutraceutical or pharmaceutical composition using one or more pharmaceutically or neutracentically acceptable excipients.

### Definitions

As used herein, the singular forms “a,” “an,” and “the” include the plural reference unless the context clearly dictates otherwise.

Reference to “about” a value or parameter herein includes (and describes) variations that are directed to that value or parameter per se. For example, description referring to “about X” includes description of “X”.

As used herein, “delaying” development of a disease includes deferring, hindering, slowing, retarding, stabilizing, and/or postponing development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of the disease is a method that reduces probability of disease development in a given time frame and/or reduces extent of the disease in a given time frame, when compared to not using the method. Such comparisons may be based on clinical studies, using a statistically significant number of subjects. Development may also refer to disease progression that may be initially undetectable and includes onset of the disease.

As used herein, the term “effective amount” intends such amount of a composition of the invention which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a composition or extract), and a single agent may be considered to be given in an effective amount if, in conjunction with one or more other agents, a desirable or beneficial result may be or is achieved. Suitable doses of any of the co-administered composition may optionally be lowered due to the combined action (e.g., additive or synergistic effects) of the composition.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The terms “individual,” “patient,” and “subject” are used synonymously, and refers to a mammal, including, but not limited to, a human.

The term “pharmaceutical composition” refers to any composition containing a biologically active component that can provide any treatment or prophylactic benefit. The term includes an adjuvant used in combination with one or more other pharmaceutical or nutracentical agents. The term “pharmaceutical” is intended to cover any biologically active nutraceutical component that provides a treatment or prophylactic benefit.

As used herein, the terms “pharmaceutically acceptable,” “pharmacologically acceptable,” or “neutraceutically acceptable” material (e.g., excipient) refer to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

A “therapeutically effective amount” refers to an amount of a compound sufficient to produce a desired therapeutic outcome.

As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease, diminishing the extent of the disease, stabilizing the disease (e.g., preventing or delaying the worsening of the disease), delaying or slowing the progression of the disease, ameliorating the disease state, decreasing the dose of one or more other medications required to treat the disease, and/or increasing the quality of life. The methods of the present disclosure contemplate any one or more of these aspects of treatment.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

Features and preferences described herein in relation to embodiments are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The disclosures of all publications, patents, and patent applications referred to herein are each hereby incorporated by reference in their entireties. To the extent that any reference incorporated by reference conflicts with the instant disclosure, the instant disclosure shall control.

### Maqui Berry Extracts and Pharmaceutical Compositions

The maqui berry extract is derived from maqui berries, but has been processed into an extract composition so that it may be more readily administered to a patient. The extract may be, for example, a dehydrated powder or concentrated liquid, which can be readily mixed with other excipients or otherwise formulated for pharmaceutical (e.g., nutraceutical) administration. The maqui berry is processed to remove portions of the liquid and/or solid components of the maqui berries. In some embodiments, the maqui berry extract is processed such that it is free or substantially free of maqui berry seed or seed pieces.

The maqui berry extract includes a high polyphenol content. Total polyphenols can be measured based on Gallic Acid Equivalents (GAE), for example using the Folin-Ciocalteu method. In some embodiments, the maqui berry extract includes at least 2%, at least 3%, at least 4%, at least 5%, at least 6%, at least 75%, at least 89%, at least 9%, at least 10%, at least 119%, at least 12%, at least 13%, at least 14%, at least 15%, at least 20%, at least 25%, at least 30%, at least 35%, at least 40%, or at least 50% total polyphenol content by weight (for example, determined as GAE equivalent, which may be determined using the Folin-Ciocalteu method). In some embodiments, the maqui berry extract includes about 2% to about 60% total polyphenol content by weight. For example, in some embodiments, the maqui berry extract includes about 2% to about 3%, about 3% to about 4%, about 4% to about 5%, about 5% to about 6%, about 6% to about 7%, about 7% to about 8%, about 8% to about 9%, about 9% to about 106%, about 10% to about 11%, about 11% to about 12%, about 12% to about 13%, about 13% to about 14%, about 14% to about 15%, about 15% to about 16%, about 16% to about 17%, about 17% to about 18%, about 18% to about 19%, about 19% to about 20%, about 20% to about 22%, about 22% to about 25%, about 25% to about 30%, about 309% to about 35%, about 35% to about 40%, about 40% to about 45%, about 45% to about 50%, or about 50% to about 60% total polyphenol content by weight.

Much of the total polyphenol content in the maqui berry extract is due to anthocyanin. Total anthocyanin in the maqui berry extract can be measured using known methods, for example a pH differential method or an HPLC method. Total anthocyanin content in the maqui berry extract may be determined using cyanidin-3-glucoside equivalents (CGE). In some embodiments, the maqui berry extract includes at least 0.5% total anthocyanin content by weight (for example, as determined by CGE). In some embodiments, the maqui berry extract includes at least 0.5%, at least 1%, at least 2%, at least 3%, at least 4%, at least 5%, at least 6%, at least 7%, at least 8%, at least 9%, at least 10%, at least 12%, at least 15%, at least 20%, or at least 25% total anthocyanin content by weight. In some embodiments, the maqui berry extract includes about 0.5% to about 30% total anthocyanin content by weight. For example, in some embodiments, the maqui berry extract includes about 0.5% to about 1%, about 1% to about 2%, about 2% to about 3%, about 3% to about 4%, about 4% to about 5%, about 5% to about 6%, about 6% to about 7%, about 7% to about 8%, about 8% to about 9%, about 9% to about 10%, about 10% to about 12%, about 12% to about 14%, about 14% to about 16%, about 16% to about 18%, about 18% to about 20%, about 20% to about 25%, or about 25% to about 30% total anthocyanin content by weight. In some embodiments, the total anthocyanin content of the maqui berry extract is about 40% to about 50% of the total polyphenol content by weight.

In some embodiments, the maqui berry extract is a powder. The powder may be fine powder, which can be mixed with other excipients to form a pharmaceutical formulation. In some embodiments, substantially all (e.g., 95% or more, 97% or more, 98% or more, or 99% or more) of the maqui berry extract can pass through a 24 mesh sieve (i.e., a sieve with an average aperture of approximately 700 micrometers).

The maqui berry extract may have a low moisture content. For example, in some embodiments, the moisture content of the maqui berry extract is less than 10% by weight. In some embodiments, the maqui berry extract has a moisture content of less than 10% by weight, less than 7.5% by weight, 5% by weight, less than 4% by weight, less than 3% by weight, or less than 2% by weight. In some embodiments, the maqui berry extract may be in a liquid or semi-solid form. For example, the maqui berry extract may have a moisture content of about 10% to about 50%, such as between about 10% and about 20%, between about 20% and about 30%, between about 30% and about 40%, or between about 40% and about 50%.

In some embodiments, the maqui berry extract includes natural maqui fiber, which may include soluble fiber, insoluble fiber, or both. In some embodiments, the maqui berry extract includes about 5% or more, about 10% or more, about 15% or more, about 20% or more, about 25% or more, about 30% or more, about 35% or more, about 40% or more, about 45% or more, or about 50% or more maqui berry fibers by weight. In some embodiments, the maqui berry extract includes about 5% to about 60% maqui berry fiber by weight (for example, about 5% to about 10%, about 10% to about 15%, about 15% to about 20%, about 20% to about 25%, about 25% to about 30%, about 30% to about 35%, about 35% to about 40%, about 40% to about 50%, or about 50% to about 60% maqui berry fiber by weight). In some embodiments, the maqui berry extract includes about 5% or more, about 10% or more, about 15% or more, about 20% or more, about 25% or more, about 30% or more, about 35% or more, about 40% or more, about 45% or more, or about 50% or more soluble maqui berry fibers by weight. In some embodiments, the maqui berry extract includes about 5% to about 60% maqui berry fiber by weight (for example, about 5% to about 10%, about 10% to about 15%, about 15% to about 20%, about 20% to about 25%, about 25% to about 30%, about 30% to about 35%, about 35% to about 40%, about 40% to about 50%, or about 50% to about 60% soluble maqui berry fiber by weight). In some embodiments, the maqui berry extract includes about 5% or more, about 10% or more, about 15% or more, about 20% or more, about 25% or more, about 30% or more, about 35% or more, about 40% or more, about 45% or more, or about 50% or more maqui berry fibers by weight. In some embodiments, the maqui berry extract includes about 5% to about 60% insoluble maqui berry fiber by weight (for example, about 5% to about 10%, about 10% to about 15%, about 15% to about 20%, about 20% to about 25%, about 25% to about 30%, about 30% to about 35%, about 35% to about 40%, about 40% to about 50%, or about 50% to about 60% insoluble maqui berry fiber by weight). In some embodiments, the maqui berry extract is a liquid extract, which may be free or substantially free (e.g., less than about 5% by weight) of insoluble maqui fiber.

The maqui berry extract may be formulated with one or more pharmaceutically acceptable excipients to form a pharmaceutical composition. The one or more pharmaceutically acceptable excipients may include, for example, one or more diluents, one or more glidant, one or more lubricants, one or more sweeteners, one or more flavoring agents, and/or one or more desiccants.

The maqui berry extract may be formulate for oral administration. Exemplary oral dosage forms can include capsules, tablets, gummies (or other chewable), or a lozenge. In some embodiments, the oral dosage form is a liquid. In some embodiments, the composition may be a powder or liquid concentrate that can be mixed with a food product for oral administration. For example, the powder may be mixed with water, juice, smoothie, cereal, yoghurt, etc., and the mixture consumed for oral administration of the maqui berry extract. For such oral administration, a pharmaceutically acceptable, non-toxic composition is formed by the incorporation of any of the normally employed excipients, such as, for example, mannitol, lactose, starch, magnesium stearate, sodium saccharine, talcum, cellulose, sodium crosscarmellose, glucose, gelatin, sucrose, magnesium carbonate, and the like. Such compositions take the form of solutions, suspensions, tablets, dispersible tablets, pills, capsules, powders, chewable dosage forms (for example, chewable tables or gummies), sustained release formulations and the like,

In some embodiments, the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose and derivatives thereof, and the like. In some embodiments, the composition contains one or more additional nutraceutical and/or bioactive components, such as one or more vitamins (e.g., vitamin A, vitamin B1, vitamin B2, vitamin B3, vitamin B5, vitamin B6, vitamin B9, vitamin B12, vitamin C, vitamin D, vitamin E, vitamin K, choline, or biotin), an essential fatty acid (e.g., an omega-3 fatty acid, or an omega-6 fatty acid), curcumin, ginkgo biloba, ginseng, turmeric, or other natural (or herbal) or synthetic supplements or medicines.

Liquid pharmaceutically administrable compositions can be prepared, for example, by dissolving, dispersing, etc. the maqui berry extract and optional pharmaceutical adjuvants or excipients in a carrier, such as, for example, water, saline, aqueous dextrose, glycerol, glycols, ethanol, and the like, to form a solution or suspension. If desired, the pharmaceutical composition to be administered may also contain minor amounts of nontoxic auxiliary substances such as wetting agents, emulsifying agents, or solubilizing agents, pH buffering agents and the like, for example, sodium acetate, sodium citrate, cyclodextrine derivatives, sorbitan monolaurate, triethanolamine acetate, triethanolamine oleate, etc. Actual methods of preparing sach dosage forms are known, or will be apparent, to those skilled in this art. The composition or formulation to be administered will contain a quantity of the active compound in an amount effective to alleviate the symptoms of the subject being treated.

The maqui berry extract or pharmaceutical composition may be packaged in a suitable container, such as a capsule, a sachet, a soft cell. The container may be a biodegradable container, which can be consumed with the pharmaceutical composition, for example for oral administration. By way of example, the container may be a biodegradable capsule, such as an HMPC or gelatin capsule. Alternatively, the capsule may be a non-biodegradable container, and the pharmaceutical formulation or the maqui berry extract can be removed from the container prior to administration.

In one example, the pharmaceutical dosage form is a chewable, such as a gummy, which can contain the maqui berry extract.

### Methods of Making Fruit or Berry Extracts

The methods described herein allow for making a maqui berry extract with a high polyphenol content. The methods may also be applied to other fruits or berries, preferably those with a high natural polyphenol content. The fruit or berry used in the methods described herein may be a single type or fruit or berry or any combination of one or more different fruits or berries in any ratio. Exemplary fruits or berries that may be processed to form an extract include, but are not limited to, blueberries, grapes, mangoes, red cherries, cranberries, blackberries, plums, pomegranates, coffee, black currents, bilberries, calafate berries (Berberis microphylla), Chilean guava (Ugni molinae), barberries (such as Berberis darwini), elderberries, and raspberries. Thus, although the method described below is provided in the context of processing maqui berries to form a maqui berry extract, the process may also be used to form extracts of other fruits or berries with high polyphenol content.

Making a fruit or berry extract with a high polyphenol content is a delicate process that can result in substantial polyphenol decay if the material is exposed to extreme temperatures for an extended period of time, particularly under high water activity conditions. While common freeze drying process allows for substantial water removal at lower temperatures, it is difficult to further dry the composition without raising the temperature of the material, which can result in polyphenol degradation. The methods described herein for making maqui berry and other fruit or berry extracts allow for substantial water removal (and, in some embodiments, removal of other solids) while minimizing the polyphenol content loss.

The fruit or berry extract described herein may be a solid or a liquid extract, The liquid extract generally has low water activity, and may be viscous at standard room temperatures. Moisture is removed, in both the solid and liquid extract forms, to reduce water activity while enhancing polyphenol stability.

The fruit or berry extract may be made by separating a fruit or berries into a polyphenol-containing liquid fraction and a fiber-containing solid fraction, and concentrating the liquid fraction to form a concentrated liquid. The fiber-containing solid fraction may also include polyphenols from the fruit or berries. That is, a proportion of the polyphenols from the fruit or berries may be distributed between the polyphenol-containing liquid fraction and the fiber-containing solid fraction. As further described herein, optionally, concentrating the liquid fraction may include applying a secondary power, such as microwave power, during a portion of the concentration step. This allows moisture to be removed from the liquid while minimizing prolonged heating. In some embodiments, no secondary power is applied to the liquid fraction, and concentration may rely on other means, such as vacuum pressure. The concentrated liquid itself may be used for the treatment methods described herein, or the concentrated liquid may be further processed. For example, in some embodiments, the method can further include combining the concentrated liquid fraction with a solid carrier to form a mixture, and drying the mixture to form the extract. Additional details for manufacturing the fruit or berry extract are described below.

In some embodiments, the fruit or berry (e.g., maqui berry) extract described herein is made by separating, from fruit or berry pulp, a polyphenol-containing liquid fraction and a fiber-containing solid fraction (which may itself further include polyphenols from the fruit or berries): concentrating the polyphenol-containing liquid fraction; mixing the concentrated polyphenol-containing liquid fraction with a solid carrier (which may be, for example, the fiber-containing solid fraction, which may be dried prior to mixing), and drying the mixture. The fruit or berry pulp may be treated, for example using ultrasonication, enzymes (such as cellulase, pectinase, or hemicellulase) and/or by applying a secondary power (such as microwave) to disrupt the fibrous material before the liquid fraction and the solid fraction are separated. In some variations of the method, however, the fruit or berry pulp is not treated to disrupt the fibrous material (i.e., not ultrasonicated, not treated with an enzyme, or not treated with a secondary power prior to separating the liquid fraction from the solid fraction). Disruption of the fibrous material affects the distribution of the polyphenols from the fruit or berry in the liquid fraction and the solid fraction. Increased disruption of the fibrous material results in a larger portion of the polyphenols in the liquid fraction. In some examples of the method, about 50% or more (e.g., about 60% or more, about 70% or more, about 80% or more, or about 90% or more, for example about 60% to about 90%, or about 70% to about 80%) of the polyphenols from the fruit or berries is in the polyphenol-containing liquid fraction. In some examples of the method, about 50% or more (e.g., about 60% or more, about 70% or more, about 80% or more, or about 90% or more, for example about 60% to about 90%, or about 70% to about 80%) of the polyphenols from the fruit or berries is in the fiber-containing solid fraction. Optionally, the liquid fractions is treated before being concentrated, for example by removing sugars from the liquid fraction. In some embodiments, sugars are not removed from the liquid fraction before concentrating.

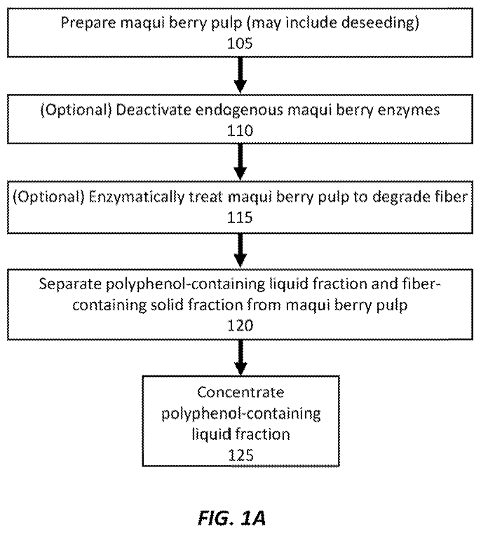

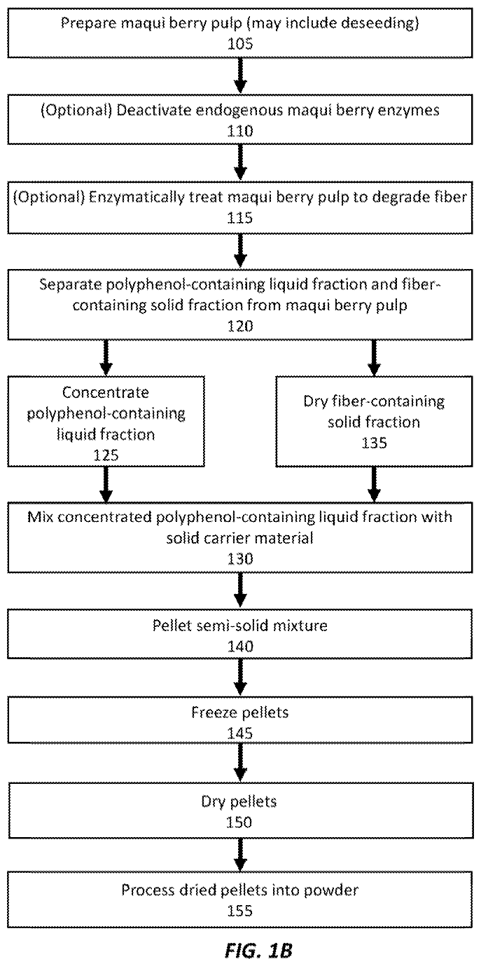

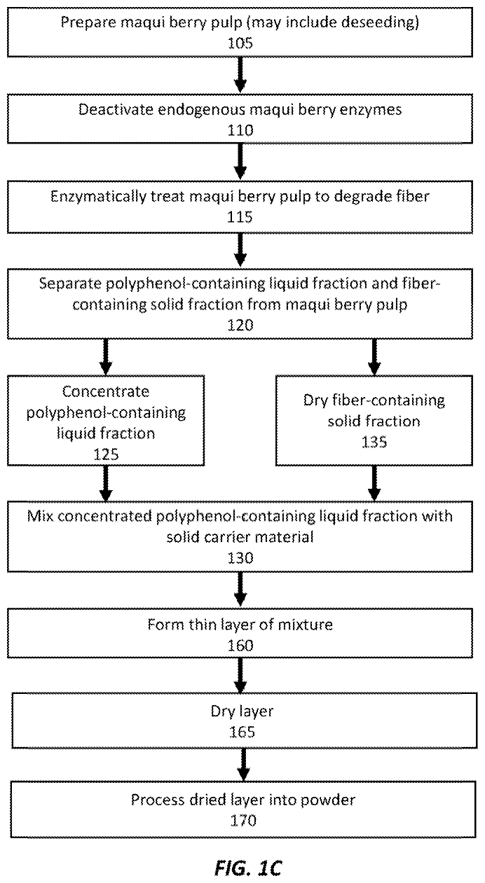

An exemplary method of making the maqui berry extract (or other fruit or berry extract) is shown in FIG. 1A. At step 105, the maqui berry pulp (or other fruit or berry pulp) is prepared. The pulp may be made using fresh, frozen, or thawed maqui berries (or other fruit or berries). In some embodiments, seeds and/or seed piece are removed from the material during the pulping process. In some embodiments, the berries or fruit are mixed with a liquid prior to pulping. The liquid may be an aqueous solution (such as water, or a buffered aqueous solution). In some embodiments, the liquid comprises a water miscible solvent, for example methanol, ethanol, or acetic acid. Exemplary buffers that may be used include, but are not limited to, citrate (or citric acid) or acetate (or acetic acid). Preferably, the liquid is acidic, for example having a pH of about 2 to about 5, about 3 to about 4, or about 3.5. The fruit or berries may be pulped at a temperature lower than room temperature (e.g., less than about 25° C., less than about 20° C., less than about 15° C., or less than about 10° C. For example, the fruit or barriers and/or liquid added to the fruit or berries may be cooled to the desired temperature. It was discovered that pulping the fruit or berries at a temperature lower than room temperature (e.g., lower than about 15° C.) result in no or minimal deterioration of the polyphenols during the pulping process. In other variants of the method, the liquid is heated, for example to about 60° C. to about 100° C., about 70° C. to about 90° C. or about 80° C. In one example, the maqui berries (or other berries or fruit) are mixed with aqueous citric acid, pH 3.5 at 80° C. In other example, the maqui berries (or other berries or fruit) are mixed with aqueous citric acid, pH 3.5 at 15° C. The maqui berries (or other berries or fruit) mixed with the liquid can be pulped in a pulper, blender, grinder, or other suitable device for breaking apart the berries or fruit in the liquid. The device may be fitted with a mesh screen, which allowed maqui berry pulp (or other berry or fruit pulp) to pass through but retains seeds and/or seed pieces. The pulp, once formed, can be chilled. Preferably, chilling occurs quickly to reduce potential polyphenol loss. Optionally, the pulp can be stored by freezing the pulp.

At optional step 110, at least a portion of the endogenous enzymes in the berries or fruit are deactivated. Active endogenous enzymes can degrade the polyphenols in the pulp. To deactivate the enzymes, the fruit or berry pulp is quickly heated before being cooled. For example, in some embodiments, the fruit or berry pulp is heated to about 60° C. to about 100° C., about 70° C. to about 90° C., or about 80° C. This process preferably occurs quickly to avoid degradation of the polyphenols, for example in less than about 10 minutes, and more preferably less than about 5 minutes. In some embodiments, the pulp is heated for less than about 2 minutes, less than about 1 minute, less than about 30 seconds, less than about 20 seconds, or less than about 15 seconds. For example, the pulp may be heated to the desired temperature for about 5 seconds to about 15 seconds. A microwave oven can be used, for example, to rapidly heat the fruit or berry pulp. Once heated, the pulp can be quickly cooled, for example by placing the pulp in a rapid chiller or submerging the pulp (in a container) in an ice-water bath, or by flash cooling (or vacuum flash cooling) the fruit or berry pulp.

FIG. 2 shows an exemplary system 200 for deactivating endogenous enzymes in the berry or fruit pulp. The system includes a conduit 202 that allows passage of the fruit or berry pulp into the system 200. A pump 204 (e.g., a peristaltic pump) can drive flow of the pulp into a microwave reaction chamber 208. A temperature sensor (e.g. a thermocouple) 206 is optionally included in the system to monitor temperature of the pulp as it enters the reaction chamber 208. The reaction chamber 208 may be, for example, a quartz tube or other tube that allows for the passage of microwaves. A magnetron 210 can emit microwaves that heat the pulp contained within the reaction chamber 208. Endogenous enzymes in the pulp are deactivated by the heating from the applied microwaves. The amount of time the pulps is exposed to microwave (and thus the heating power) depends on the length of the reaction chamber and the flow rate, as controlled by the pump 204. A temperature sensor (e.g., a thermocouple) 212 can be included in the system to monitor temperature of the pulp as it exits the reaction chamber. The desired temperature is controlled by the amount of power applied by the magnetron and the exposure time of the pulp in the reaction chamber (e.g., as controlled by the pump rate). By way of example, the pulp may be heated to a temperature of about 60° C. to about 100° C., about 70° C. to about 90° C., or about 80° C. The microwaves may be applied, for example, for about 5 seconds to about 30 seconds, such as about 5 seconds to about 15 seconds, or about 10 seconds. The reaction chamber 208 is fluidly connected to cooling chamber 216 through 214. As the pulp is pumped out of the reaction chamber 208, it enters the cooling chamber 216 where it quickly cools. The cooling chamber 216 is under a vacuum pressure (e.g., about 5 mbar to about 30 mbar, or about 10 mbar to about 20 mbar, or about 15 mbar). The pulp is cooled due to water evaporation in the cooling chamber 216, and the steam can be removed from the cooling chamber 216 through a vacuum exhaust 218. A pressure sensor 220 can be included to monitor pressure within the cooling chamber. The pulp may be cooled in the cooling chamber to about 10° C. to about 25° C. The cooled pulp may then exit the system through a conduit 224 by being pulled from the cooling chamber 216 by pump (e.g., a peristaltic pump) 222.

At optional step 115, the fruit or berry (e.g., maqui berry) pulp is enzymatically treated to at least partially degrade fiber in the fruit or berry (e.g., maqui berry) pulp. For example, the maqui berry pulp may be treated with cellulase, pectinase, or hemicellulase. Treating the fruit or berry pulp to partially degrade fiber in the fruit or berry pulp results in an increased portion of the polyphenols to be present in the liquid fraction. This step is optionally, as in some embodiments, it is desired to retain the polyphenols in the fiber-containing solid fraction.

At step 120, a polyphenol-containing liquid fraction is separated from a fiber-containing solid fraction. Optionally, prior to separating the liquid and solid fractions, the pulp is blended to reduce fiber size. Also optionally, energy may be applied to the pulp, which can help separate or degrade the fibers in the pulp. The energy may be, for example, ultrasonic energy, microwave energy (such as at about 950 MHz or 2450 MHZ), or a pulsed electric field (PEF). The liquid and solid fractions of the pulp may be separated, for example by centrifuging the pulp. In some embodiments, the liquid and solid fractions are separated by countercurrent extraction. Separation of the liquid and solid fractions may occur in one or more (e.g., 1, 2, 3, 4 or more) iterations. For example, the pulp may be centrifuged, the liquid fraction removed, and the solid fraction re-suspended (for example, by blending) in fresh liquid (e.g., the same or a different type of liquid used when pulping the fruit or berries). In some embodiments, a countercurrent extraction is used, and a first removed fraction is used to wash the solid fraction.

The solid fraction the liquid fraction may be separated by pressing the pulp. Pressing may be preferred, for example, when pulp does not undergo further processing to degrade fiber. The temperature may be controlled during the pressing process. For example, in some embodiments, the temperature of the solid faction and/or liquid fraction may be maintained below room temperature (e.g., at about −20° C. to about 20° C., about −10° C. to about 20° C., about 0° C. to about 20° C. or about 15° C.). The applied pressure during pressure may be, for example, about 1 bar to about 5 bars, about 2 bars to about 4 bars, or about 3 bars. Pressing separates the liquid fraction of the pulp from the solid fraction of the pulp. For example, prior to pressing the pulp (which has not been processed to degrade fibers) may include about 25% total solids, but pressing the pulp (for example, about 3 bars at about 15° C.) separates the pulp into a liquid fraction (which can include about 55% to about 60% of the total pulp weight, with about 25% soluble solids and about 20% to about 35% total polyphenols) and the solid fraction (which can include about 40% to about 45% of the total pulp weight, with about 40% total solids, and about 65% to about 80% total polyphenols). The resulting solid fraction may be a pressed cake with about 60% to about 65% moisture content.

Optionally, the liquid fraction is processed before concentrating the liquid fraction at step 125. The liquid fraction may be processed, for example, to remove a portion or all of the sugars in the liquid fraction. This may be accomplished, for example, using a membrane separation method. For example, a 200 Dalton nanofiltration membrane may be used to reduce the sugar content of the liquid fraction. In another example, a cationic resin may be used to remove sugars from the liquid fraction.

At step 125, the polyphenol-containing liquid fraction is concentrated. Concentration of the polyphenol-containing liquid fraction preferably occurs under temperature-controlled or room. temperature conditions. Controlling the temperature during concentration prevents excessive heating that can result in polyphenol degradation. In some embodiments, the temperature of the polyphenol-containing liquid fraction during concentration about 60° C. or less, about 50° C. or less, about 40° C. or less, about 30° C. or less, about 25° C. or less, or about 20° C. or less. In some embodiments, the polyphenol-containing liquid fraction is maintained as a liquid (e.g., about 0° C. or higher, such as about 10° C. or higher, or about 20° C. or higher) during the concentration process. In some embodiments, the temperature increases during the concentration process as the liquid fraction becomes more concentrated, preferably staying below a maximum temperature (which may be set, for example, at a temperature of about 60° C. or less, about 50° C. or less, about 45° C. or less, about 40° C. or less, about 45° C. or less, or about 30° C. or less). Temperature adjustments during the concentration process are further discussed herein. Concentration can occur by evaporating water (or other solvent, such as methanol or ethanol, if used in the liquid fraction). In some embodiments, concentration of the polyphenol-containing liquid fraction occurs under vacuum. For example, the vacuum pressure may be about 4000 Pa or less (such about 500 Pa to about 3000 Pa, or about 1000 Pa to about 2000 Pa). Exemplary methods of concentrating the polyphenol-containing liquid fraction include, but are not limited to, rotary evaporation, falling film evaporation, thin film evaporation, circulation evaporation, rising film evaporation, climbing and falling-film plate evaporation, or other suitable evaporation methods. In some embodiments, the polyphenol-containing liquid fraction is evaporated using a rotary evaporator.

The temperature of the liquid fraction may be carefully controlled during the concentration step. Polyphenol instability is closely associated with moisture content and temperature of the composition. Thus, as the moisture content of the liquid fraction decreases (and, in turn, the water activity (a_{w}) decreases), the temperature of the liquid fraction may be increased to further evaporate the water. By initiating the concentration process at a lower temperature, the time the composition is exposed to elevated temperatures (and thus, the risk of polyphenol degradation) is reduced. The temperature may be ramped or stepwise increased from an initial starting temperature (for example, about 30° C. or less, about 25° C. or less, or about 20° C. or less) to a maximum temperature (for example, a maximum temperature of about 60° C. or less, about 50° C. or less, about 45° C. or less, about 40° C. or less, about 45° C. or less, or about 30° C. or less). For example, concentration of the liquid fraction may be initiated under vacuum pressure at a temperature between about 0° C. and about 20° C., or between about 20° C. and about 30° C., and proceed for a period of time (e.g., about 10 minutes to about 20 minutes) during an initial concentration phase. During the initial concentration phase, the moisture content of the liquid fraction may decrease to about 50%, or a water activity between about 0.88 and 0.9. The temperature can then be increased, for example to between 30° C. and about 40° C. (such as about 35° C.), and the liquid fraction can continue to concentrate until the moisture content decreases to about 35%, or a water activity of about 0.8. The temperature can then again be increased to between 40° C. and about 50° C. (such as about 45° C.), and the liquid fraction can continue to concentrate until the moisture content decreases to about 20%, or a water activity of about 0.7. These temperatures, moisture contents, and water activities are exemplary, and other suitable temperatures and concentration times may be used.

In some embodiments, a secondary power (such as a microwave power) is applied to the liquid fraction during part or all of the concentration process at step 125. The secondary power is a power source other than the vacuum, such as microwave, infrared, temperature differential radiation, direct conduction, or other secondary power source. The secondary power source may heat the liquid fraction. Application of the secondary power source refers to actively applying power to the liquid fraction and is intended to convey the application of power beyond the vacuum or any ambient power or energy (e.g., thermal energy or lighting in the room surrounding any device used to concentrate the liquid fraction of dry some other composition). For example, concentration may proceed during a first step, wherein the liquid fraction is concentrated (for example, under vacuum pressure) without applying a secondary power, and then during a second step, wherein the liquid fraction is further concentrated (for example, under vacuum pressure) with a secondary power (such as microwave power) being applied. Moisture can be removed during the initial concentration step (e.g., without applying the secondary power) until the water activity or moisture content of the liquid fraction is reduced to a target value or until no further moisture (or no significant moisture) may be removed without applying the secondary power. The temperature of the liquid composition during this first step may be lower than the second concentrating step, which limits degradation of the polyphenols. The temperature of the liquid fraction may be increased during the second step by virtue of the applied secondary power. The increased temperature (and applied secondary power) allows additional moisture to be removed from the liquid fraction. The amount of time at the elevated temperature may be limited to minimize degradation of the polyphenols in the liquid composition.

In one example, the liquid fraction may be concentrated in a continuous evaporator. Exemplary continuous evaporators include a thin film evaporator, a wiped film evaporator, or a falling film evaporator. Evaporation in the continuous evaporator may occur under vacuum pressure. An exemplary method of continuous evaporation can include continuously flowing the liquid fraction into a chamber under vacuum pressure and, simultaneously, removing concentrated liquid fraction from the chamber. Liquid in the chamber may be warmed, for example by circulating the liquid through a heater. Evaporation due to the vacuum pressure can cause a decrease in temperature, which may be compensated for by the heater. The pressure of the chamber may be, for example, less than about 50 mbar (such as less than about 40 mbar, less than about 30 mbar, or less than about 20 mbar, such as about 10 mbar to about 20 mbar). The temperature of the liquid may maintained, for example, at a temperature above freezing, for example about 10° C. to about 30° C. (such as about 20° C. to about 30° C., or about 25° C. to about 28° C.). Polyphenols in the liquid fraction have increased stability when the soluble solids content is about 50% or higher, while the un-concentrated liquid fraction may have, for example, a soluble solids content of about 20% to about 25%. Thus, the liquid fraction may be concentrated in the chamber until the soluble solids content is about 50% or more (e.g., about 55% or more, about 60% or more, about 65% or more, or about 70%, such as about 55% to about 80%, or about 55% to about 65%, or about 65% to about 75%). Once the soluble solids content of the liquid fraction in the chamber reaches the desired amount, liquid fraction can be added to the chamber as concentrated liquid fraction is removed to reach an approximately steady state of soluble solids of the liquid in the chamber. The average residence time of liquid in the chamber may be, for example about 20 seconds to about 20 minutes (such as about 20 seconds to about 40 seconds, about 40 seconds to about 1 minute, about 1 minute to about 5 minutes, about 5 minutes to about 10 minutes, about 10 minutes to about 15 minutes, or about 15 minutes to about 20 minutes).

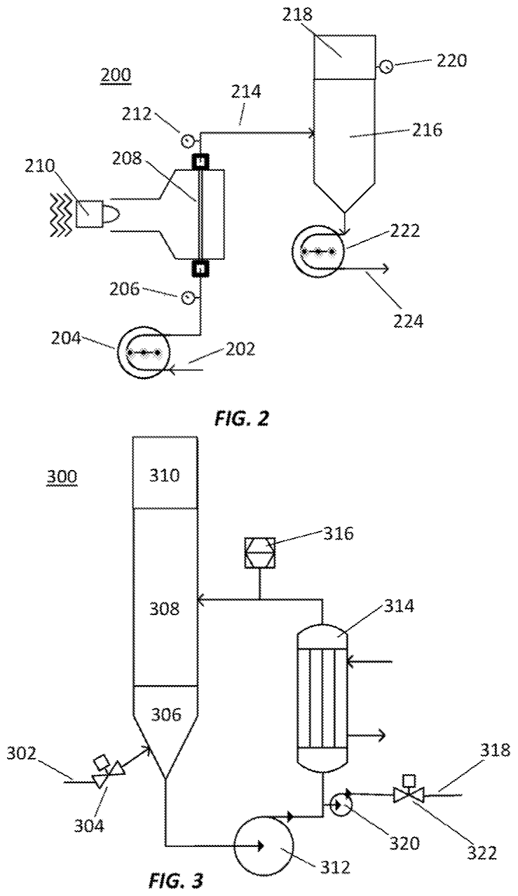

FIG. 3 shows an exemplary continuous evaporator for concentrating the liquid fraction in accordance with some embodiments of the method described herein. Un-concentrated liquid fraction can continuously flow through the system inlet 302 into the evaporation chamber 308. A valve 304 can be included to control the flow rate of the liquid fraction entering the evaporation chamber 308. The evaporation chamber 308 may be held under vacuum pressure using a vacuum 310. The vacuum pressure speeds evaporation of liquid from the liquid fraction 306 in the evaporation chamber 308, and the evaporated liquid can be exhausted through a vent of the vacuum 310. The liquid fraction 306 can be heated, for example by circulating the liquid fraction 306 through a heater 314 (e.g., a heat exchanger). A pump 312 (e.g., a peristaltic pump) can be used to circulate the liquid fraction from the evaporation chamber 308, through the heater 314, and back to the evaporation chamber 308. Optionally, the soluble solids content of the liquid fraction 306 may be monitored using a sensor 316 (e.g., a refractometer), which is optionally disposed in line with the recirculation conduit. The system can include an outflow 318, wherein concentrated liquid fraction is continuously removed from the continuous evaporator. A pump 320 can be used to pull concentrated liquid fraction, optionally in line with the recirculation conduit. A valve 322 may also be included to control outflow rate. To initiate the continuous evaporator, an initial amount of un-concentrated liquid fraction enters the evaporation chamber through the inlet. A vacuum pressure is applied in the evaporation chamber as the initial liquid fraction is circulated through the heater. Once the desired soluble solids content of the liquid fraction is reached (e.g., about 50% soluble solids or more, about 55% soluble solids or more, about 60% of soluble solids or more, or about 65% soluble solids or more), the continuous evaporator can enter a steady state, wherein the solids content of the liquid fraction in the evaporation chamber is approximately stable. Un-concentrated liquid fraction flows into the evaporation chamber as concentrated liquid fraction is simultaneously removed from the continuous evaporator system.

In some embodiments, the concentrated liquid fruit or berry extract is used in accordance with the treatment and/or formulation methods described herein. In some embodiments, the concentrated liquid fraction is further processed into a dried composition. For example, the liquid fraction may be combined with a solid carrier (for example, a dried fiber-containing solid fraction) to form a mixture, which may then be dried (e.g., by freeze-drying) to form the extract For example, the liquid fraction may be processed using the exemplary method provided in FIG. 1B or FIG. 1C.

In an exemplary method of making a maqui berry extract, the method can include separating maqui berries (which may be fresh, frozen, or thawed (i.e., previously frozen and thawed) into a polyphenol-containing liquid fraction and a fiber-containing solid fraction. Although the liquid fraction contains polyphenols, about 50% or more of the polyphenols from the maqui berries may be in the solid fraction (for example, if the fibers in the solid fraction are not degraded prior to being separated from the liquid fraction). Separating the maqui berries into the liquid fraction and the solid fraction can include pulping the maqui berries to form a maqui berry pulp, and optionally deactivating endogenous enzymes in the maqui berry pulp (for example by heating the pulp). The liquid fraction may be concentrated, for example using a continuous concentrator, to form a concentrated liquid fraction. Optionally, sugars in the liquid fraction may be removed prior to concentrating the liquid fraction. In parallel, the solid fraction may be dried. For example, the solid fraction may be dried under a vacuum pressure, such as by freeze-drying the solid fraction. The concentrated liquid fraction can then be combined with the dried solid fraction to form a mixture. The mixture may then be dried, for example by freeze-drying, to form the maqui berry extract.

Referring now to the exemplary method described in FIG. 1B (which incorporates the method described with respect to FIG. 1A), the concentrated polyphenol-containing liquid fraction is mixed with a solid carrier material at step 130. The solid carrier material may be a dried material (for example, a material having a moisture content of about 16% or less, about 15% or less, about 14% or less, about 13% or less, about 12% or less, about 11% or less, about 10% or less, about 9% or less, about 8% or less, about 7% or less, about 6% or less, about 5% or less, about 4% or less, about 3% or less, or about 2% or less). In some embodiments, the moisture content of the solid carrier material is less than the moisture content of the concentrated polyphenol-containing liquid fraction. In some embodiments, the moisture content of the solid carrier material is about 1% to about 5%. Exemplary solid carrier materials can include mannitol, lactose, glucose, maltose, glycine, trehelose, sorbitol, starch, magnesium stearate, sodium saccharine, talcum, cellulose, sodium crosscarmellose, glucose, gelatin, sucrose, magnesium carbonate, gelatin, and the like. In some embodiments, the solid carrier material is a powder.

In some embodiments, the solid carrier material is or is derived from the fiber-containing solid fraction of the maqui berry pulp. For example, in some embodiments, the fiber-containing solid fraction of the maqui berry pulp is dried to reduce the moisture content (see optional step 135) before being mixed with the polyphenol-containing liquid fraction. The fiber-containing solid fraction can be dried, for example, in an oven and/or under a vacuum (such as in a freeze-drying process or vacuum dram dryer). In some embodiments, the fiber-containing solid fraction is dried at a temperature of about 50° C. to about 90° C., or about 60° C. to about 80° C. or about 70° C. The solid fraction may alternatively be dried under a reduced temperature (e.g., between about −30° C. and about 30° C., about −20° C. and about 20° C., about −10° C. and about 30° C., about 5° C. and about 20° C., or about 10° C. and about 15° C., or about 10° C.) under vacuum (for example, at a pressure of about 0.5 mbar to about 1 mbar). The fiber-containing solid fraction may be spread into a thin layer (e.g., having a thickness of about 10 mm or less, or about 6 mm or less) and dried until the moisture content is sufficiently reduced. This drying process may take, for example, several hours, such as about 1 to about 3 hours, or about 2 hours, depending on the desired moisture content, the temperature of the oven, and the thickness of the material. In some embodiments, the solid carrier material is a powder. For example, the dried fiber-containing solid fraction, or other solid carrier material, may be milled to a suitable size.

The concentrated polyphenol-containing liquid fraction can be mixed with the solid carrier material (which may be, for example, all of or a portion of the dried fiber-containing solid fraction) to form a semi solid material (i.e., having a dough-like consistency). The moisture content of this material is generally about 30% or less, such as about 25% or less, about 20% or less, or about 18% or less. The mixture may then be further dried and processed into a powder, for example as described below according to the exemplary methods illustrated in FIG. 1B and FIG. 1C.

The semi-solid mixture can be pelleted at step 140. In some embodiments, the pellets are formed using a grinder, such as a meat grinder. Other suitable devices for forming pellets are known in the art. In some embodiments, the pellets have a diameter between about 1 mm and about 10 mm, for example between about 2 mm and about 8 mm, about 4 mm and about 6 mm, or about 5 mm.

At step 145, the pellets in step 140 are frozen. The pellets may be flash-frozen, for example by mixing the pellets with liquid nitrogen (or other inert super-cooled liquid) or placing the pellets in a container which is submerged in liquid nitrogen (or other inert super-cooled liquid). In some embodiments, the pellets are placed in a cold storage to freeze. The pellets may be cooled, for example, to a temperature lower than about −50° C., lower than about −60 ° C., lower than about −70° C., lower than about −80° C., lower than about −90° C., lower than about −100° C. lower than about −120° C., or lower than about −140° C. Freezing the pelleted material is advantageous particularly when the drying step, further described at step 150, includes mixing or tumbling the mixture during the drying process. Mixing or tumbling the pellets without freezing could cause the pellets to agglomerate, which, if a secondary power is applied during the drying process, could prevent penetration of the secondary power and inconsistent drying.

At step 150, the frozen pellets are dried by simultaneously applying a vacuum and a secondary power to the frozen pellets. In some embodiments, the frozen pellets are mixed or tumbled during the drying process. For example, a microwave freeze-dryer may include a device to tumble the pellets under vacuum while the microwave power is applied. In some embodiments, the pellets are allowed to warm while being dried, although preferably the temperature of the pellets does not exceed a maximum desired temperature. The maximum desired temperature may be, for example, about 50° C. or less, about 40° C. or less, about 35° C. or less, about 30° C. or less, or about 25° C. or less. In some embodiments, the temperature of the pellets does not exceed about 10° C. about 15° C., or about 20° C. for more than about 30 minutes, more than about 45 minutes, or more than about 60 minutes. Step 150 may proceed as two or more steps, for example, a first step at a lower temperature and a second step at a high temperature (which may be below the maximum desired temperature). The energy or power applied to the pellets may be, for example, an infrared energy, temperature differential radiation, direct conduction, or a microwave energy. In some embodiments, the energy is a microwave energy. Exemplary methods for microwave-assisted freeze drying are discussed in Zhang et al., Trends in microwave-related drying of fruits and vegetables, Trends in Food Science & Technology, vol. 17, pp. 524-534 (2006). In some embodiments, the microwave energy is pulsed during the drying process. The temperature of the material can be monitored during the process and the frequency and length of the microwave pulses can be adjusted based on the temperature to ensure the temperature does not exceed a desired maximum. In some embodiments, such as when the energy is applied by microwave, the microwave energy may be applied at a frequency of about 950 MHz or about 2450 MHz, or a combination thereof. Drying of the pellets occurs relatively quickly, for example over the course of less than 6 hours, less than 5 hours, less than 4 hours, or less than 3 hours. The pellets may be dried until a low moisture content is reached, for example a moisture content of less than about 5%, less than about 4%, or less than about 3%. In some embodiments, the pellets are dried until they have a moisture content between about 1% and about 5%, or between about 2% and about 4%.

At step 155, the dried pellets are processed into a powdered fruit or berry (e.g. maqui berry) extract, for example by milling or grinding the material. In some embodiments, the pellets are processed in batches. In some embodiments, the powdered extract is passed through a mesh screen.

Although the extract manufacturing processing may include pelleting the mixture, freezing the pellets, and then drying the frozen pellets, other methods of drying the mixture formed at step 130 are contemplated. FIG. 1C shows another exemplary method of making a maqui berry extract, which incorporates steps 105 to 135 as described in reference to FIG. 1A and FIG. 1B. Referring to FIG. 1C, at step 160, the semi-solid mixture is spread into a thin layer, for example a layer having a thickness of about 5 mm or less. In some embodiments, the layer has a thickness of about 1 mm to about 2 mm, about 2 mm to about 3 mm, about 3 mm to about 4 mm, or about 4 mm to about 5 mm. The mixture may be spread into a thin layer on a plate, such as a freeze-drier plate or other suitable tray that can be configured to receive energy. Optionally, the layer is frozen, but it is not necessary to freeze the layer according to the example provided in FIG. 1C.

At step 165, the layer containing the mixture is dried by simultaneously applying a vacuum and a secondary power to the layer. The tray holding the layer may be configured to receive the secondary power, which can be transferred to the layer, for example by induction. In some embodiments, the secondary power applied to the layer may be, for example, an infrared energy, temperature differential radiation, or direct conduction. In some embodiments, the layer is allowed to warm while being dried, although preferably the temperature of the layer does not exceed a maximum desired temperature. The maximum desired temperature may be, for example, about 50° C. or less, about 40° C. or less, about 35° C. or less, about 30° C. or less, or about 25° C. or less. In some embodiments, the temperature of the layer does not exceed about 10° C., about 15° C., or about 20° C. for more than about 30 minutes, more than about 45 minutes, or more than about 60 minutes. Step 160 may proceed as two or more steps, for example, a first step at a lower temperature and a second step at a high temperature (which may be below the maximum desired temperature). The temperature of the material can be monitored during the process and the frequency and amount of secondary power can be adjusted based on the temperature to ensure the temperature does not exceed a desired maximum. Drying of the layer occurs relatively quickly, for example over the course of less than 6 hours, less than 5 hours, less than 4 hours, or less than 3 hours. The layer may be dried until a low moisture content is reached, for example a moisture content of less than about 5%, less than about 4%, or less than about 3%. In some embodiments, the layer is dried until they have a moisture content between about 1% and about 5%, or between about 2% and about 4%.

At step 170, the dried layer is processed into a powdered fruit or berry (e.g. maqui berry) extract, for example by milling or grinding the material. In some embodiments, the layer or portions of the layer are processed in batches. In some embodiments, the powdered extract is passed through a mesh screen.

The fruit or berry (e.g., maqui berry) extract can be formulated into a pharmaceutical composition may mixing the extract with one or more pharmaceutically acceptable excipients. In some embodiments, the pharmaceutical composition is a powdered composition. The pharmaceutical composition may be packaged into a suitable container, such as a capsule, sachet, or bottle. In some embodiments, the extract is formulated into a chewable formulation, such as a gummy.

### Treatment of Neurodegenerative Disease

Maqui berry extracts, such as those described herein and/or manufactured according to the methods described herein, and composition containing such maqui berry extracts, can be used for treating or preventing (or delaying the onset of) a neurodegenerative disease. In some embodiments, the maqui berry extract is administered as an adjuvant in combination with one or more additional therapeutic agents effective in treating the disease.

In one embodiment, the subject is a human who is considered to be at risk for developing a neurodegenerative disease (such as Alzheimer's disease or Parkinson's disease), for example, an individual who has one or more biomarkers for the disease. In one embodiment, the individual is a human who is genetically predisposed to developing the neurodegenerative disease.

In one embodiment, the subject is a human who has a neurodegenerative disease (such as Alzheimer's disease or Parkinson's disease).

In some embodiments, the neurodegenerative disease is Alzheimer's disease, prion disease, stroke, multiple sclerosis, Parkinson's disease, Huntington's disease, memory loss, Creutzfeldt-Jakob disease, amyotrophic lateral sclerosis (ALS) disease, Pelizaeus-Merzbacher disease, a cognitive impairment, or dementia.

In some embodiments, the neurodegenerative disease is Alzheimer's disease. In some embodiments, the neurodegenerative disease is Parkinson's disease.

In some embodiments, the maqui berry extract is administered with one or more additional therapeutic agents effective for treating the disorder. For example, in some embodiments, the maqui berry extract is used to treat Alzheimer's disease by administering the maqui berry extract to the patient in combination with a therapeutically effective amount of a therapeutic agent effective for treating Alzheimer's disease. Exemplary therapeutic agents that may be used for treating Alzheimer's disease include a cholinesterase inhibitor, such as an acetylcholinesterase inhibitor, or an NMDA receptor antagonist. In some embodiments, the therapeutic agents used for treating Alzheimer's disease in combination with the maqui berry extract is one or more of tacrine, rivastigmine, galantamine, donepezil, memantine, solanezumab, or aducanumab.

### EXAMPLES

The application may be better understood by reference to the following non-limiting examples, which are provided as exemplary embodiments of the application. The following examples are presented in order to more fully illustrate embodiments and should in no way be construed as limiting the scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1. Manufacture of a Maqui Berry Extract

25 kg of frozen cleaned maqui berries were obtained from a local producer in Chile. The frozen maqui berries were characterized as having a soluble solid of 24.5 Brix, (optic, temperature-balanced refractometer) and total moisture of 58.5%. Total polyphenol content of the frozen berries was measured as 8720 mg of Gallic Acid Equivalents (GAE) per 100 gram of dry berries (Folin-Ciocalteu Method). Total anthocyanin of the berries was measured as 3628 mg of cyanidin-3-glucoside equivalent per 100 gram of dry berries (total anthocyanin method). The ratio of anthocyanin to polyphenols was therefore determined to be 0.416. In manufacturing the maqui berry extract, total polyphenols were used as the main variable to validate a gross polyphenol retention.

The frozen berries were mixed with 7 liter of an aqueous citric acid solution at pH 3.5 at 80° C. and stirred. The mixture was pulped in a pilot size pulper equipped with a double pallet rotor of a dimeter of 0.2 m and a 1 mm perforated screen, operated at 800 rpm. Seeds were successful separated from the pulp material during the pulping process. 20.5 kg of seedless maqui berry pulp was recovered, measured at 16.75 Brix and 77% moisture content (determined using a thermo-hygrometer at 115° C.). Liquid phase total polyphenols were measured to be 16,008 mg of Gallic Acid Equivalents per 100 grams of dry pulp (Folin-Ciocalteu Method), and anthocyanin content was measured to be 7,341 mg per 100 grams of dry pulp.

The maqui berry pulp (10.5 liters) was poured into polylaminated bags and sealed before being submerged in a water-ice bath. The polylaminated bags were needed in the ice-water bath for 5 minutes to reduce the temperature to less than 10° C. in 5 minutes before storing the pulp at −20° C.

The maqui berry pulp was then heated in 500-gram batches in an open bag placed in a 1000 ml beaker using a commercial microwave oven (700 watt) to reach 80° C. in about 4 minutes and 20 seconds. The heated pulp was kneading to homogenize the material before submerging the sealed bag in an ice-water bath for 35 seconds, reaching a temperature of 10° C. No polyphenol loss was detected during this procedure. The chilled maqui berry pulp was then blended for 1 minute in a blender (33,000 rpm) in 1-liter batches to reduce the fiber size.

The blended maqui berry pulp was centrifuged in 1 kg batches for 5 minutes at 8000 rpm. The liquid was decanted from the centrifuge bottle, obtaining 6.45 kg of a liquid fraction 17.41 Brix) and a 2.42 kg solid fraction. The solid fraction was re-suspended in an aqueous citric acid solution, pH 3.5, to reach a 5 kg mass and homogenized using a hand blender for 60 seconds. The blended material was centrifuged, and the liquids decanted to obtain a 3.95 kg second liquid fraction (5.22 Brix). The first and second liquid fraction were mixed obtaining a total of 11.45 kg of liquid (13.2 Brix). Total polyphenols were measured in the liquid as 21,529 mg GAE per 100 grams of dry sample.

The solid fraction was dispersed on a tray in a 5 mm layer and dried in a convection oven at 70° C. for 2 hour, resulting in a dried carrier material weighing 1.2 kg having a moisture content of 5.2%.

The liquid fraction was concentrated using a rotary evaporator. In 500 mL batches, the liquid fraction was loaded into a 1500 mL evaporation flask. Vacuum was applied to reach a pressure of about 2000 Pa with the evaporation flask partially submerged in a 30° C. bath. The rotary evaporator was equipped with a cooling system using a 2° C. cooling fluid. Temperature of the concentrating liquid was taken every 3 minutes using an infrared thermometer. 11.11 kg of liquid fraction (with an estimate 11.8% dissolved solids) was concentrated to 1.69 kg of liquid (with an estimated 72% dissolved solids. The concentrated liquid was stored at 4° C. Total polyphenol content of the concentrated liquid fraction was determined to be 21,059 mg GAE per 100 grams of dry sample content, representing a loss 5.8% from the liquid fraction prior to concentration.

The concentrated liquid fraction was mixed with the dried solid fraction and homogenized in a mixer to obtain a 2.75 kg mixture having 18% moisture content. The mixture was pelleted using a meat grander into pellets of approximately 5 mm in diameter at 5° C.

The pelleted material was dried using a microwave-assisted freeze-drying process, 550-gram batches of the pellets were frozen and placed in a 50-liter turntable. Pressure was reduced to 2 Pa, and increased to 5 Pa during the application of a microwave pulse. Microwave power was applied in 200-watt pulses as 2450 MHZ. The material was allowed to dry for 2 hours and 20 minutes, resulting in a dried material with 2.8% moisture. Total polyphenol content was measured as 14,630 mg of GAE per 100 grams of dried sample content.

The dried material was then ground into a powder using a grinder operated at 10,000 rpm for 8 seconds, in 200-gram batches.

### Example 2. Maqui Berry Extract Reduces Amyloid-β Aggregation

Aggregation of amyloid-β (Aβ₁₋₄₀) was tested in PBS buffer with 20 μM of Thioflavin T (ThT, Sigma-Aldrich). The wells further include no maqui berry extract (control) or 0.81 mg/L or 0.081 mg/L of a maqui berry extract. The plate was kept at room temperature with an orbital agitation of 500 rpm. Thioflavin T-Aβ complex was measured using fluorescence (excitation: 440 nm, emission: 485 nm) every 3 min for 4 h. Thioflavin T binds to Aβ aggregates, and a higher fluorescent signal indicate more Aβ aggregate formation. Results are shown in FIG. 4 (MBX=maqui berry extract). Wells without maqui berry extract showed a strong fluorescence signal, showing substantial Aβ aggregate formation. When the samples were treated with maqui berry extract, however, Aβ aggregation decreased. Larger amounts of maqui berry extract reducing the amount of Aβ aggregation.

### Example 3. Maqui Berry Extract Increases Cell Viability in Presence of Amyloid B

Cellular health was quantified using a 3-(4,5-dimethylthizol-2-yl)-2,5-diphenyltetrazolium bromide (MTT) assay, which indicates cell viability. Cultured PC12 cells were incubated in MTT (1 mg/ml) in 1X DPBS (Dulbecco's phosphate-buffered saline) for 30 minutes, in the presence (or absence) of maqui berry extract provided a concentrations ranging from 0.000081 mg/l to 81 mg/l. Precipitated MTT was dissolved using isopropanol for 15 minutes prior to being added to the culture medium. The cell absorbance was measured in a microplate reader (Novostar, BGM, Germany) at two wavelengths: 560 nm and 620 nm. The difference in cellular absorbance was quantified between the two wavelengths using NovoStar software across experimental conditions.

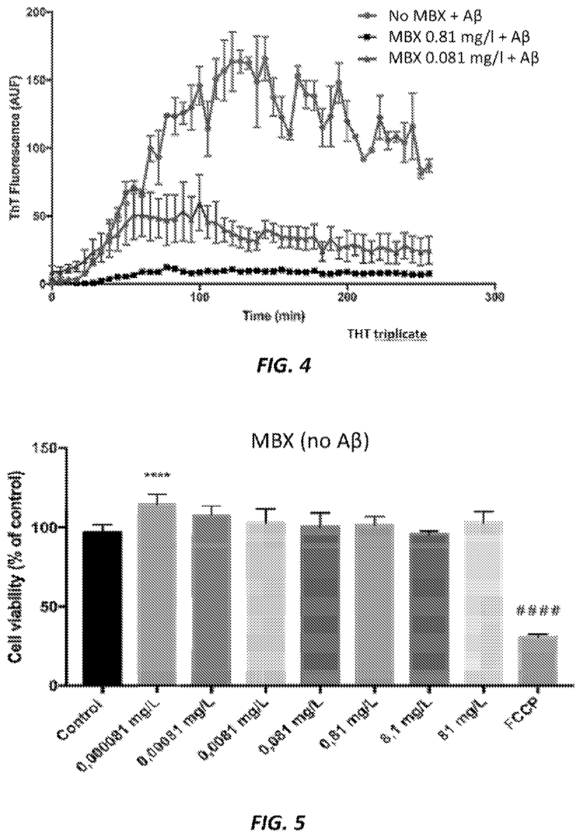

As shown in FIG. 5, increasing concentrations of maqui berry extract did not have cytotoxic effects on cells in culture. This indicates that cells preserve functionality and redox capability in the presence of maqui berry extract. Further, the addition of Carbonyl cyanide 4-(trifluoromethoxy)phenylhydrazone (FCCP) had a detrimental effect on cell viability, and caused a significant decrease in cell viability percent relative to control levels.

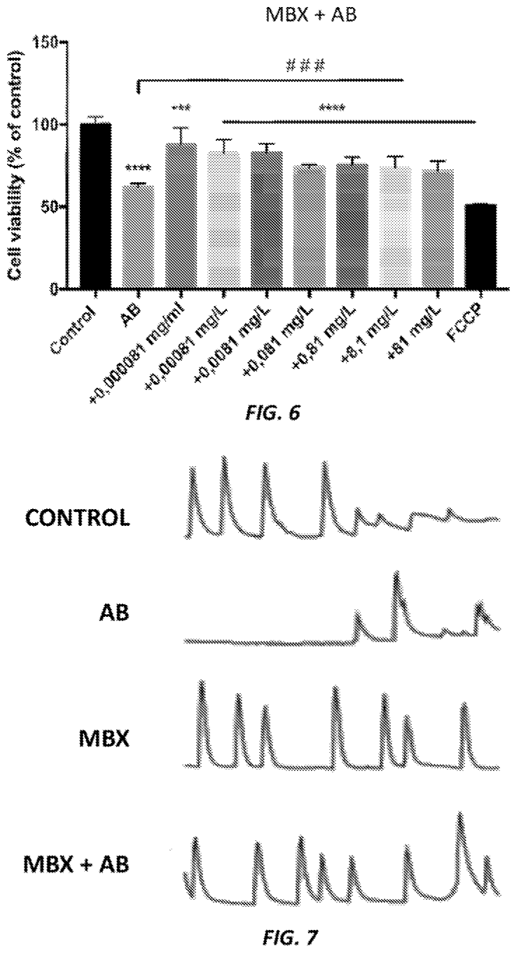

Next, the ability of maqui berry extract to counteract the cytotoxic effects of Aβ in culture was tested. Cultured PC12 cells were incubated in MTT (1 mg/mL) in IX DPBS (Dulbecco's phosphate-buffered saline) for 30 minutes, in the presence (or absence) of a maqui berry extract provided a concentrations ranging from 0.000081 mg/l to 81 mg/l and the presence (or absence) of 0.5 μM Aβ₁₋₄₀. Aβ negatively impacted cell viability, while the presence of the maqui berry extract mitigated some of the cytotoxic effects (FIG. 6).

### Example 4. Maqui Berry Extract Maintains Electrophysiological and Structural Properties of Cultured Neurons Exposed to Amyloid-Beta

Fluorescence measurements of calcium transients were performed to determine whether maqui berry extract can preserve homeostatic neuronal signaling. A fluorescent calcium indicator was added to a neuronal hippocampal culture treated with (1) 0.5 μM Aβ₁₋₄₀ peptides for 24 hours, (2) a maqui berry extract (0.081 mg/L) for 24 hours, or (3) 0.5 μM Aβ₁₋₄₀ peptides and a maqui berry extract (0.081 mg/L) for 24 hours. Cell cultures were incubated in 1× DPBS solution containing 5 μM of the fluorescent probe Fluo-4AM, a cell-permeable dye used to measure calcium concentration in cells, (Invitrogen, Carlsbad, CA, USA) for 20 minutes at 37° C. Subsequently, a wash was performed with DPBS for 20 minutes to remove any excess Fluo-4AM probes. Afterward, two additional washes were performed with standard external solution. Next, intracellular calcium (Ca²⁺) transients were recorded using an inverted fluorescent NIKON TE-2000 microscope (Tokyo, Japan) coupled to a 16-bit EM-CCD iXon+camera (Andor, Belfast, North Ireland). Images were recorded every 1 second with a time exposure of 200 ms. The images were analyzed using Imaging WorkBench 6.0 software (INDEC Biosystems, Santa Clara, CA, USA).

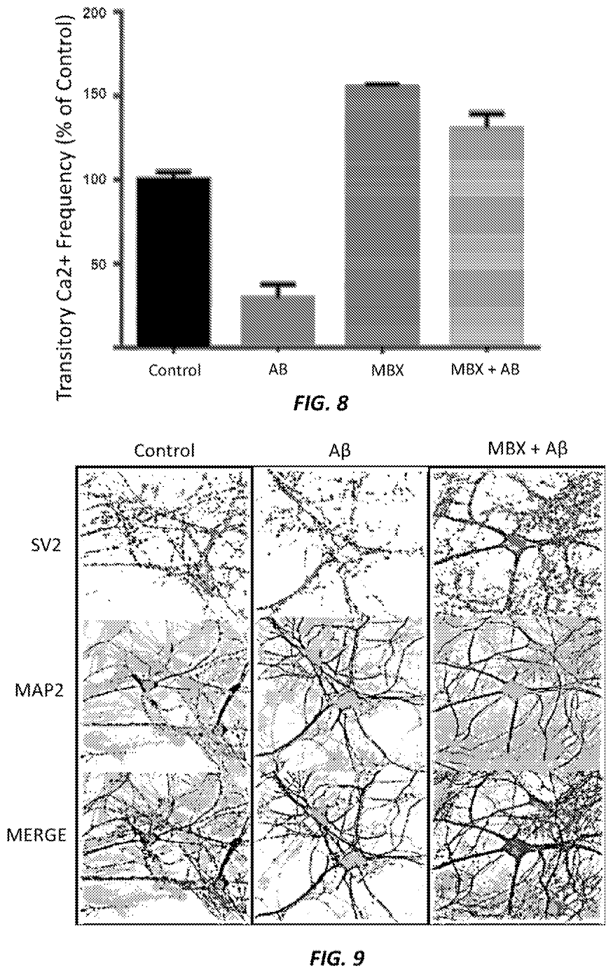

FIG. 7 shows neuron synaptic frequency measures for Ca²⁺ systolic transient recordings in a hippocampal neural array. The control trace (FIG. 8, top trace) shows the synaptic frequency in a standard hippocampal neural array and FIG. 8 shows the control transitory Ca²⁺ frequency (normalized to control) in a 200-millisecond period. In the presence of Aβ, the intensity and rate of synaptic transients was reduced, as indicated by synaptic silence (FIG. 7, second trace). The synaptic silence cause by the presence of Aβ is also seen in FIG. 8. When incubated in 0.081 mg/l of maqui berry extract, neurons showed an increase in the frequency of calcium transients (FIG. 7, third trace; and FIG. 8). Further, incubating the neurons with maqui berry extract muted the toxic effects of Aβ, and the frequency of calcium transients was even increased relative to control (FIG. 7, bottom trace; and FIG. 8).

### Example 5. Neuroprotective Effect of Maqui Berry Extract

To test whether maqui berry affected the total amount of synaptic connections within cultured neurons, an immunohistological staining experiment was performed. Hippocampal neuron arrays were exposed, for 24 hours, to (1) 0.5 μM Aβ₁₋₄₀ peptides, (2) 0.5 μM Aβ₁₋₄₀ peptides with a maqui berry extract, or (3) neither (control). Cell cultures were washed with 1× PBS and fixed in 4% paraformaldehyde in PBS for 15 minutes at room temperature. Cells were then incubated in a blocking buffer containing 10% horse serum and permeabilized with 0.1% Trixon-X-100 for five minutes. Synaptic vesicle glycoprotein 2 (SV2) was detected using a specific SV2 antibody (anti-mouse SV2 diluted to 1:200, Hybridoma Bank). Neuronal structure was identified using the microtubule-associated protein 2 (MAP2) antibody (anti-rabbit MAP2 diluted to 1:100, Santa Cruz). Cultures were incubated in the primary antibody solution for a total of 1 hour at room temperature. Next, cultures were incubated in a secondary antibody solution containing Cy3 (anti-mouse Cy3 diluted to 1:200, Jackson ImmunoResearch Laboratories) and AlexaFluor (anti-rabbit diluted to 1:200, Abcam) to label SV2 and MAP2, respectively. The secondary antibody incubation was performed for 45 minutes at room temperature. Cells were mounted with Dako Fluorescent Mounting Medium (Dako, USA). Images were obtained with a spectral confocal microscope (LSM780 NLO Zeiss) in the Center of Advanced Microscopy (CMA) and analyzed with ImageJ software.

FIG. 9 shows the immunohistochemistry images (sharpness, contrast, and brightness adjusted, converted to greyscale) of the cells stained for SV2 or MAP2, and merged images. The control hippocampus cell culture array shows healthy cell development and synapses. Images of the cell array treated with Aβ shows decreased cell count, body engrossment, and a reduction of synapses. Cell arrays treated with both Aβ and maqui berry extract has a healthy body structure and a significant increase in synapses. These results correlates with the synaptic frequencies measured in calcium transient experiments.

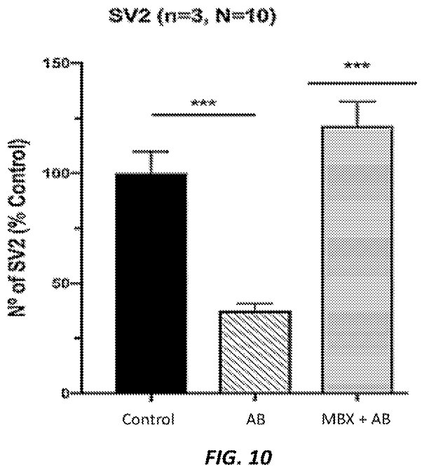

SV2 was quantified along the axon of individual neurites. The percentage of SV2 puncta relative to controls was compared for control. Aβ treated neurons, and maqui berry and Aβ treated neurons. Neurons exposed to Aβ treatment had a significant reduction in the total amount of SV2 expression relative to control neurons. In contrast, application of Aβ with the maqui berry extract resulted in a significant increase in SV2 expression relative to Aβ treatment (FIG. 10). This result further demonstrated the neuroprotective effect of maqui berry extract over the toxic beta amyloid peptides.

### Example 6. Maqui Berry Extracts Improve Memory in Mice

The effects of maqui berry extract on memory were tested in aged C57 mice in a Barnes Maze experimental paradigm. Aged C57 mice were fed 4 mg of maqui berry extract once a day, 5 days a week, for a total of 19 days. The behavioral equipment was cleaned with ethanol prior to each trial. The maze was set-up in a quiet testing room with a temperature range of 20-24° C. Prior to testing, mice were placed into the dark testing room in their original cages for an acclimation period lasting for 20 minutes. Mice were trained by being placed into the center of the Barnes Maze, with the lights on, and guided to their cages. For each training session, the mice needed to remain in their cage for at least one minute after finding it from the center of the maze.

Training was quantified by recording the spatial movements of mice within the maze with a camera. The video was collected for a total of three minutes or when the mouse enters the safety cage, whichever came first. This was repeated for a total of 7 trials for each mouse with an inter-trial-interval of 15 minutes. Video was recorded and analyzed using Anymaze software.

Short-term memory testing was performed 24 hours after training was completed for each mouse. As before, the maze was kept dark until testing was ready to begin. Individual mice were placed in the center of the Barnes Maze, and the recording equipment and light were turned on. The latency to reach the maze and the trajectory of each mouse taken to reach the maze were extracted from the video. Long-term memory testing was performed similarly to short-term memory testing except experiments were started 12 days after the last training session of each mouse.

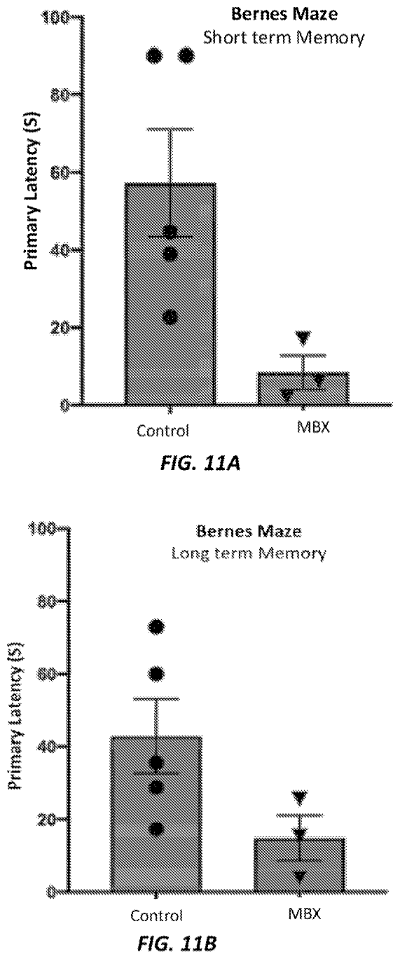

In short-term memory testing of aged control mice (n=5), the latency to reach the safety cage was found to be around 60 seconds on average (FIG. 11A). Mice fed maqui berry extract (n=3) had a markedly reduced latency to find the safety cage, which was around 10 seconds on average for the three mice tested. For long-term memory, aged control mice showed a latency to reach the safety cage of 40 seconds, whereas the aged maqui berry extract-fed mice took an average of around 15 seconds to reach the safety cage (FIG. 11B). These results indicate that maqui berry extract fed aged mice performed better in short- and long-term memory tests than aged mice fed a standard diet.

### Example 7. Manufacture of a Maqui Berry Extract

30 kg of frozen cleaned maqui berries, characterized in having 25.6 Brix and 3128 mg of cyanidin-3-glucoside equivalent per 100 gram of berries (dry weight), as determined by the total anthocyanin method, where thawed at room temperature for 20 hours to reach a temperature of approximately −1.4° C. The thawed berries were pulped and seeds removed, resulting in a seedless pulp having 25.4% soluble solids, 6% insoluble fiber, and 72% moisture.

Pulp was treated by microwave power to deactivate endogenous enzymes, using the device shown in FIG. 2. Briefly, pulp was loaded with a variable flow peristaltic pump at an input temperature of 25° C. and heated by a 1 kW microwave magnetron, reaching 82° C. in 12 seconds. The microwave-treated pulp was flash cooled at 20 mbar absolute pressure, reaching 22° C.

The pulp was pressed by applying a 2.2 bar pressure with a 150 micron filter, separating the pulp into a liquid fraction (58% by weight) with 23% soluble solids and a fiber-containing solid fraction with 62% moisture. Approximately 73% of the polyphenols in the pulp were in the solid fraction.

The solid fraction was spread into a 3-4 mm layer on a beatable surface and freeze-dried. The solid fraction was frozen and a 0.2 mbar vacuum applied, while the heatable surface was heated. After 2 hours and 57 minutes, the solid fraction had a 7% moisture content.

The liquid fraction was concentrated using a continuous evaporator as shown in FIG. 3. Vacuum pressure of the evaporation chamber was set to 7 mbar, with liquid within the evaporation chamber held at approximately 23° C. and 65% solids content.

The dried solid fraction and concentrated liquid fraction were mixed together in a dough mixer at 20° C. resulting in a mixture having 19% moisture content. The mixture was then spread into a 3 to 3.5 mm layer on a heatable surface and freeze dried for 3 hours and 24 minutes, reaching a 4.8% moisture content. The freeze-dried composition was milled at room temperature to obtain a 35-mesh extract powder, with a total anthocyanin content of 4875 mg canidin-3 glucoside equivalent per 100 grams of dry extract.

The determination of the anthocyanin content was made using the method established by Tanaka et al., Maqui berry (Aristotelia chilensis) and the constituent delphinidin glycoside inhibit photoreceptor cell death induced by visible light, Food Chemistry, vol. 139, pp. 129-137 (2003), with some modifications. The powdered extracts were mixed with a water acid solution and centrifuged at 14,000 rpm for 10 min and injected into a Water™ ALLIANCE 2695 with Photo Diode Array Detector Waters 2996 and Software Empower®. Chromatographic conditions were as follows; a XTerra C18 WaterTM Column (250×5 mm) was used, the column furnace temperature was 30° C. The mobile phase included 0.3% trifluoroacetic acid (A) and acetonitrile (B): 0-4th min 95% acetonitrile; 4.5th min 90% acetonitrile; 27th min 85% acetonitrile; 47th min 45% acetonitrile; 48th min 10% acetonitrile ; 50th min 10% acetonitrile; 51st min 95% acetonitrile and 60th min 95% acetonitrile, with a flow rate of 0.7 mL/min. The detection wavelength was selected at 520 nm. The injection volume of the maqui extract was 20 μL. Peaks were identified based on their ultraviolet-visible spectra, co-chromatography respect to commercial standards. when available, and by comparison with elution order as reported in other published studies. Anthocyanin distribution for the raw material (i.e., the maqui berry) and the final powered extract powder is shown in Table 1.

TABLE 1

| | | Maqui Berry |
|---|---|---|
| Anthocyanin | Maqui Berry | Extract |
| Delphinidin-3-sambubioside-5 glucoside | 36.10% | 33.78% |
| Delphinidina-3.5-diglucoside | 25.60% | 18.11% |
| Cyanidin-3.5-diglucoside | 8.88% | 15.30% |
| Cyanidin-3-sambubioside 5-glucoside | 4.65% | 4.83% |
| Delphinidin 3-sambubioside | 6.18% | 7.80% |
| Delphinidin 3 glucoside | 13.60% | 11.83% |
| Cyanidin 3 sambubioside 5 glucoside | 2.21% | 2.93% |
| Cyanidin 3 glucoside | 2.79% | 5.41% |

1. A method of making a fruit or berry extract, comprising:
separating a fruit or berries into a polyphenol-containing liquid fraction and a fibercontaining solid fraction;
concentrating the liquid fraction to form a concentrated liquid fraction;
combining the concentrated liquid fraction with a solid carrier to form a mixture; and drying the mixture to form the extract.

2. The method of claim 1, wherein the fruit or berries are maqui berries.

3. The method of claim 1, wherein the fiber-containing solid fraction comprises polyphenols from the fruit or berries.

4. The method of claim 3, wherein fiber-containing solid fraction comprises about 50% or more of the polyphenols from the fruit or berries.

5. The method of claim 1, wherein the polyphenol-containing liquid fraction comprises about 50% or more of the polyphenols from the fruit or berries.

6. The method of claim 1, wherein the solid carrier comprises a portion or all of the fiber-containing solid fraction.

7. The method of claim 6, comprising drying the solid fraction prior to mixing the concentrated liquid fraction with the solid carrier.

8. The method of claim 7, wherein the solid fraction is dried under a vacuum pressure.

9. The method of claim 7, wherein the solid fraction is dried at a temperature between about −30°C. and about 30° C.

10. The method of claim 1, wherein concentrating the liquid fraction comprises applying a vacuum and a secondary power to the liquid fraction.

11. The method of claim 10, wherein the secondary power applied to the liquid fraction comprises microwave power.

12. The method of claim 10, wherein the secondary power is heat conduction.

13. The method of claim 1, wherein the liquid fraction is maintained at a temperature of about 10° C. to about 30° C. while being concentrated.

14. The method of claim 1, wherein the liquid fraction is concentrated using a continuous evaporator that simultaneously receives the liquid fraction and outflows the concentrated liquid fraction.

15. The method of claim 14, wherein liquid fraction in the continuous evaporator has a soluble solids content of about 50% to about 80%.

16. The method of claim 1, comprising removing seeds or seed pieces prior to separating the fruit or berry prior to separating the fruit or berries into the liquid fraction and the solid fraction.

17. The method of claim 1, comprising pulping fruit or berries to form fruit or berry pulp, wherein the liquid fraction and the solid fraction are formed from the fruit or berry pulp.

18. The method of claim 17, comprising deactivating endogenous enzymes in the fruit or berry pulp.

19. The method of claim 18, wherein deactivating the endogenous enzymes comprises heating the fruit or berry pulp to a temperature between about 60° C. to about 100° C., and cooling the fruit or berry pulp.

20. The method of claim 17, comprising applying an ultrasonic, microwave energy, or an electromagnetic energy to the pulp.

21. The method of claim 17, comprising mixing the fruit or berry pulp with one or more enzymes that at least partially degrade fiber in the fruit or berry pulp.

22. The method of claim 21, wherein the one or more enzymes comprises one or more of cellulase, pectinase, and hemicellulase.

23. The method of claim 1, comprising pelleting the mixture prior to drying the mixture.

24. The method of claim 1, comprising forming a layer comprising the mixture, wherein the layer has a thickness of about 1 mm to about 5 mm.

25. The method of claim 1, comprising freezing the mixture prior to drying the mixture.

26. The method of claim 1, wherein drying the mixture comprises applying a vacuum and a secondary power to the mixture.

27. The method of claim 26, wherein the secondary power is applied using infrared, temperature differential radiation, direct conduction, or microwave power.

28. The method of claim 27, wherein the secondary power is applied using microwave power.

29. The method of claim 1, comprising:
pelleting the mixture to form a pelleted mixture;
freezing the pelleted mixture to form a frozen, pelleted mixture; and
drying the frozen, pelleted mixture by applying a vacuum and microwave power to the frozen, pelleted mixture, to form the extract.

30. The method of claim 1, comprising:
forming a layer comprising the mixture, wherein the layer has a thickness of about 1 mm to about 5 mm; and
drying the layer comprising the mixture by applying a vacuum and a secondary power to the layer, to form the extract.

31. The method of claim 30, wherein the secondary power is applied using infrared, temperature differential radiation, or direct conduction.

32. The method of claim 26, wherein the secondary power is applied at about 0.05 kW to about 0.25 KW per kg of the mixture.

33. The method of claim 1, wherein the temperature of the mixture increases to a maximum temperature of 50° C. or less during the drying.

34. The method of claim 1, wherein the temperature of the mixture increases as the water activity of mixture decreases during the drying.

35. The method of claim 1, wherein the temperature of the mixture does not exceed 30° C. for more than about 30 minutes during the drying.

36. The method of claim 1, wherein the liquid fraction is concentrated at a temperature between about 20° C. and about 35° C. when the water activity of the liquid fraction is about 0.75.

37. The method of claim 1, wherein the liquid fraction is concentrated at a temperature between about 30° C. and about 45° C. when the water activity of the liquid fraction is between about 0.6 and about 0.75.

38. The method of claim 1, wherein the fruit or berries are separated into the liquid fraction and the solid fraction using centrifugation or counter-current extraction.

39. The method of claim 1, wherein the fruit or berries are separated into the liquid fraction and the solid fraction by pressing fruit or berry pulp.

40. The method of claim 1, wherein the mixture is dried until it has a moisture content of about 5% or less by weight.

41. The method of claim 1, comprising processing the extract into a powder.

42. A method of making a liquid fruit or berry extract, comprising:
separating a fruit or berries into a polyphenol-containing liquid fraction and a fibercontaining solid fraction; and
concentrating the liquid fraction, comprising applying a vacuum and a secondary power to the liquid fraction, to form a concentrated liquid fruit or berry extract.

43. The method of claim 42, wherein the fruit or berries are maqui berries.

44. The method of claim 42, wherein the secondary power applied to the liquid fraction comprises a microwave power.

45. The method of claim 42, wherein the secondary power applied to the liquid fraction is heat conduction.

46. The method of claim 42, wherein the liquid fraction is maintained at a temperature of about 10° C. to about 30° C. while being concentrated.

47. The method of claim 42, wherein the liquid fraction is concentrated using a continuous evaporator that simultaneously receives the liquid fraction and outflows the concentrated liquid fraction.

48. The method of claim 47, wherein liquid fraction in the continuous evaporator has a soluble solids content of about 50% to about 80%.

49. The method of claim 42, wherein concentrating the liquid fraction comprises:
a first concentrating step, comprising applying the vacuum to the liquid fraction without applying the secondary power to the liquid fraction; and
a second concentrating step, comprising applying the vacuum and the secondary power to the liquid fraction.

50. A fruit or berry extract made by the method of claim 1.

51. The method of claim 1, comprising formulating the extract with one or more pharmaceutically acceptable or neutracetucially acceptable excipients.

52. A composition made according to the method of claim 51.

53. The composition of claim 52, wherein the composition is a pharmaceutical composition or a nutraceutical composition.

54. A maqui berry extract comprising at least 2% total polyphenol content by weight, at least 10% maqui berry fiber by weight, and a moisture content of less than 5% by weight.

55. The maqui berry extract of claim 54, comprising at least 10% total polyphenol content by weight.

56. The maqui berry extract of claim 54, comprising about 14% to about 40% total polyphenol content by weight.

57. The maqui berry extract of claim 54, wherein the extract comprises at least 25% maqui berry fiber by weight.

58. The maqui berry extract of claim 54, wherein the maqui berry fiber comprises insoluble maqui berry fiber.

59. The maqui berry extract of claim 54, wherein the maqui berry fiber comprises soluble maqui berry fiber.

60. The maqui berry extract of claim 54, wherein the maqui berry extract is a powder.

61. The maqui berry extract of claim 54, wherein the maqui berry extract is substantially free of maqui seeds or maqui seed pieces.

62. A composition comprising the maqui berry extract of claim 54 and a pharmaceutically acceptable or neutraceutically acceptable excipient.

63. A pharmaceutical or nutraceutical dosage form, comprising the composition of claim 62, wherein the dosage form is formulated as an oral dosage form.

64. The pharmaceutical dosage form of claim 63, wherein the oral dosage form is a chewable dosage form or a capsule.

65. The pharmaceutical dosage form of claim 64, wherein the oral dosage form is a chewable gummy.

66. A method of treating a neurodegenerative disease in a subject, comprising administering to the subject a therapeutically effective amount of the extract according to claim 50.

67. A method of preventing or delaying the onset of a neurodegenerative disease in a subject, comprising administering to the subject a therapeutically effective amount of the extract according to claim 50.

68. The method of claim 66, further comprising administering to the subject a therapeutically effective amount of an additional therapeutic agent for treating, preventing, or delaying the onset of the neurodegenerative disease.

69. The method of claim 66, wherein the neurodegenerative disease is Alzheimer's disease or Parkinson's disease.

70. A method of increasing memory of a subject, comprising administering to the subject an effective amount of the extract according to claim 50.
